(19) United States
(12) Patent Application Publication
(10) Pub. No.: US 2004/0047882 A1
(43) Pub. Date: Mar. 11, 2004

(54) Adjuvant for vaccines

(76) Inventor: Michael Broeker, Marburg (DE)

Correspondence Address: Alisa A Harbin, Chiron Corporation, Intellectual Property R338, PO Box 8097, Emeryville, CA, 94662-8097, US

(21) Appl. No.: 10/221,941

(22) PCT Filed: Mar. 14, 2001
(86) PCT No.: PCT/EP01/02866

(30) Foreign Application Priority Data
Mar. 14, 2000 (DE) 10012370.

(51) Int. Cl. A61K 48/00; A61K 39/145; A61K 31/739

(52) U.S. Cl. 424/206.1; 514/54; 514/44

(57) ABSTRACT

Vaccine containing a first vaccine, adjuvated with an oil-in-water emulsion comprising 5% squalene, 0.5% polysorbate 80 and 0.5% sorbitan trioleate in aqueous citrate buffer pH 6.5, and a nonadjuvated second vaccine as combination partners for the simultaneous, separate or phased application for immunization against viral, bacterial or parasitic infectious diseases.

The invention involves the use of an oil-in-water emulsion as an adjuvant to be applied contralaterally. The invention especially involves vaccines containing a first vaccine, adjuvated with an oil-in-water emulsion, and a second vaccine, not adjuvated with this adjuvant, as combination partners for the simultaneous, separate or phased application for therapy or prophylaxis. The invention very especially involves combinations of an influenza vaccine, adjuvated with MF59, and a second vaccine.

Numerous vaccine formulations containing attenuated pathogens or protein subunit antigens have so far been developed. Conventional vaccine preparations usually contain adjuvants to strengthen the immune response. For example, depot-forming adjuvants are frequently used, which absorb and/or precipitate the administered antigen and form a depot at the injection site. Typical depot-forming adjuvants include aluminum compounds (alum) and water-in-oil emulsions. However, although depot-forming adjuvants increase the antigenicity, they frequently cause severe, persistent local reactions such as granulomas, abscesses and cicatrices if they are applied subcutaneously or intramuscularly.

On injection, other adjuvants such as lipopolysaccharides and muramyl dipeptides can cause pyrogenic reactions or Reiter's syndrome with flu-like symptoms, generalized arthralgia and sometimes also anterior uveitis, arthritis and urethritis. Saponins, such as those from Quillaja saponaria, have likewise been used as adjuvants in vaccines.

MF59, an immunostimulating submicron oil-in-water emulsion having a safe application, has recently been developed for use in vaccine formulations, see e.g. Ott et al., “MF59Design and Evaluation of a Safe and Potent Adjuvant for Human Vaccines” in Vaccine Design: The Subunit and Adjuvant Approach (Powell, M. F. and Newman, M. J., editors) Plenum Press, New York, 1995, pages 277-296. So far only aluminum salts and MF59 have been licensed for use as adjuvants for formulating vaccines for application in humans.

Adjuvants can act in various ways; they can influence the cytokine network, direct antigens to potent antigen-presenting cells, induce cytotoxic T-lymphocytes or prolong the release of the antigen via depot formation. The conventional application of adjuvants and vaccines usually takes place at the same time and place so as to increase an immune response to the applied antigen.

For MF59 a temporal and spatial separation of the application of antigen and adjuvant in an animal experiment has been described, although without specific data on the various application sites (Dupuis et al., Vaccine 18 (2000), 434-439, Dupuis et al., Cellular Immunology 186 (1998), 18-27 and Ott et al., “MF59-Design and Evaluation of a Safe and Potent Adjuvant for Human Vaccines: in Vaccine Design: The Subunit and Adjuvant Approach (Powell, M. F. and Newman, M. F., editors) Plenum Press, New York, 1995, pages 277-296), which nonetheless resulted in an increase in the applied immunity/antigenicity of the temporally and/or spatially separate antigens. However, a (simultaneous) contralateral application of MF59 or a vaccine, adjuvated with MF59, in combination with a second vaccine, not adjuvated with MF59, has not yet been described.

The invention in question is based on the surprising and unexpected discovery that the spatially separate consecutive or simultaneous application of MF59 or of a vaccine, adjuvated with MF59, produces a synergistic effect on the antigenicity/immunogenicity of a second vaccine, not adjuvated with MF59, in humans.

Based on the mode of action of MF59 discussed in the literature, this effect should not be expected. It should thus be noted that the mechanism of action for MF59 has not yet been thoroughly clarified.

Although a stimulation of cytokine synthesis, especially of IL-5 and IL-6, has been discussed (e.g. Cellular Immunology, 186 (1998), pages 18-27), it has been shown in particular that MF59 affects the recruiting and activation of antigen-presenting cells such as dendritic cells in muscle, for example, which take up the antigen, migrate to the draining lymph nodes and efficiently present the processed antigen to the T-lymphocytes, which should at least suggest that a certain spatial proximity to the application site of adjuvant or antigen should be present in the muscle. As mentioned above, although the spatially separate application of MF59 and antigen in an animal experiment resulted in an adjuvation (stimulation of antigenicity/immunogenicity), the effects found with the contralateral application in humans are even more amazing if one considers—as the specialist is adequately aware—that it is not possible to extrapolate the results obtained with adjuvants in an animal experiment involving small mammals, in particular, to large mammals, not to mention humans. This should be taken into consideration, especially for contralateral application, since spatial separation in small mammals is of course not as obvious.

The contralateral simultaneous application of the two vaccines, one of which is adjuvated with MF59 and the other is not adjuvated with MF59, is the preferred embodiment of the invention in question. “Contralateral,” as used in the description in question and in the claims, is defined as the application on opposite sides of the body, such as e.g. usually in the deltoid (musculus deltoides) of the right and left arm.

The application can take place consecutively or simultaneously, simultaneous application being preferred.

The oil-in-water emulsion preferably used as adjuvant is MF59, whose composition and preparation is described as follows:

MF59

1. squalene (2, 6, 10, 15, 23-hexamethyl-2, 6, 10, 14, 18, 22-tetracosahexane), about 5% (39 mg/ml)

2. polysorbate 80 (Tween® 80), approx. 0.5% (4.7 mg/ml)

3. sorbitan trioleate 85 (Span® 85), approx. 0.5% (4.7 mg/ml)

4. citrate buffer pH 6.5 (trisodium citrate dihydrate, citric acid monohydrate, water for injection)

MF59 is prepared in a per se known manner (Ott et al., “MF59-Design and Evaluation of a Safe and Potent Adjuvant for Human Vaccines” in Vaccine Design: The Subunit and Adjuvant Approach (Powell, M. F. and Newman, M. J., editors) Plenum Press, New York, 1995, pages 277-296).

Polysorbate 80 is dissolved in water for injection and sodium citrate buffer is added. Sorbitan trioleate is dissolved in squalene separately. These two solutions are combined, and an emulsion is prepared in a homogenizer (microfluidizer). After filtration through a 22 μm filter and removal of larger drops under nitrogen treatment, the result is a milky, white, stable emulsion, which contains essentially particles having a diameter <1.2 μm. The resulting emulsion can be admixed to the vaccine to be adjuvated either while the vaccine is being prepared or shortly before it is applied, such as e.g. in the formulation with the recombinant surface glycoprotein gp120 of human immunodeficiency virus (HIV), to prevent changes in confirmation. A proximal application of antigen and MF59 is also possible.

“Vaccine,” as used in the description and the claims, is defined as viral, bacterial or parasitic antigens. They can exist in the form of whole-cell viruses, bacteria, parasites, protein subunits, polysaccharides, polysaccharide conjugates and nucleic acids. They can be used without modification in galenic form or in combination with vehicles or carriers such as e.g. microspheres, liposomes, nanospheres, ISCOMS and other antigen delivery systems familiar to the specialist.

As already mentioned above, an especially preferred embodiment of the invention is the combined simultaneous contralateral application of an influenza protein subunit vaccine adjuvated with MF59, such as Fluad® with a nonadjuvated capsule polysaccharide vaccine against Streptococcus pneumoniae. The contralateral simultaneous application of these two vaccines is especially advantageous because the patient group for whom the inoculation with both vaccines is recommended is for the most part mutually overlapping. A group inoculation was recommended by the St\check{S}ndige Impfkommission des Robert-Koch-Institut [Permanent Vaccination Committee of the Robert Koch Institute] (STIKO), especially for immunosuppressed patients (e.g. immunosuppression caused by high-dose steroid treatment, condition after transplantations, dialysis patients) and special risk groups such as diabetics and nursing home residents. This patient group in particular is not only especially at risk of an influenza infection, but also has an increased risk of pneumococcus infection. Bacteria of the species Streptococcus pneumoniae are the most frequent pathogens of purulent bronchitis and bacterial lung infection. Other severe pneumococcal diseases are acute purulent meningitis, acute endocarditis, sepsis and peritonitis. Pneumococcal pneumonia has a mortality rate of 10%, and risk factors present in the aforementioned patient group increase the mortality rate to 20-30%. After age 50 the mortality rate is even higher.

Viral flu or influenza in humans is an acutely febrile infectious disease which usually appears as an epidemic and can quickly spread across continents as a pandemic. Infection with influenza viruses normally occurs during the winter months. Three different types of influenzavirus are known: influenzavirus A, B and C. Influenza viruses are RNA viruses and are members of the Orthomyxoviridae family. The influenzavirus has a complex construction. It consists of a filamentous ribonucleic capsid which is surrounded by a shell. The antigens hemagglutinin (HA) and neuraminidase (NA) are integrated on the outside of the shell. These two antigens sit on the particle surface like fungiform spikes. HA and NA are important for the adhesion and intracellular penetration of the virus. For the influenzavirus that can infect humans, three HA serotypes (H1, H2 and H3) and two NA types (NA1 and NA2) are known. Extensive preclinical and clinical studies have shown that HA is capable of inducing protective, virus-neutralizing antibodies.

Influenzavirus is distinguished by a genetic peculiarity: The viral ribonucleic acid (RNA) is divided into eight segments, which can be passed on separately to the viral progeny. This makes possible an arbitrary new combination among viral particles of a virus type. Virus type A is subject to the phenomenon of antigen change via antigen drift and antigen shift. Antigen drift is defined as a point mutation in the HA gene. New drift variants are responsible for the appearance of epidemics. Antigen shift is defined as the exchange of larger gene segments between different animal and human influenza strains (reassortment of RNA segments). In 1957 the surface antigens H1N1 developed into H2N2 via exchange of homologous RNA segments between human and animal influenzavirus strains, and in 1968 H2N2 developed into H3N2. Viral flu is a highly contagious disease occurring throughout the world, which is typically caused pandemically by type A, epidemically by type B and only sporadically by type C.

Epidemics with influenza A and B result in high infection rates, especially in the preschool and school age. Adults who live in contact with small children are susceptible to an especially high risk of becoming ill. Diseases caused by influenzavirus A have a moderate to severe course and affect all population groups. Persons with chronic diseases of the cardiovascular system and respiratory tract, metabolic dysfunction, immune dysfunction and kidney diseases are at an especially high risk. Persons with congenital heart defects also have an especially high risk after an infection with influenza viruses.

Effective vaccines are available for prevention. Three different types of vaccines are offered: deactivated whole-particle, split and subunit vaccines. In Germany only split and subunit vaccines are currently offered. These influenza vaccines contain highly purified, split and deactivated virus particles, the subunit vaccines containing only the virus-specific surface antigens HA and NA and the split vaccines additionally containing viral matrix proteins. The vaccines contain the antigens of one representative of each influenzavirus type which is established annually by WHO for the pertinent vaccine of the season in question. These are currently one influenzavirus A strain each of formula H3N2 and H1N1 and also one strain of influenzavirus B.

According to a “Note for Guidance on Harmonization of Requirements for Influenza Vaccines” of the Committee for Proprietary Medicinal Products of the European Agency for Evaluation of Medicinal Products, the minimum requirements have been standardized with respect to the composition and potency of influenza vaccines, and all influenza vaccines contain e.g. 15 μm HA of each of the three strains per vaccine dose. The effectiveness of a flu inoculation in healthy adults is over 75%. In people over 60 and in immunosuppressed persons, the protection rate is considerably lower. According to estimates of the Arbeitsgemeinschaft Influenza [Influenza Task Force], in Germany alone about 5000 to 10,000 people, mostly persons from high-risk groups, die from influenza.

Numerous attempts have been made to increase the protective effect of influenza vaccines, especially in high-risk groups, via addition of adjuvants. One of the most commonly used adjuvants for human vaccines are aluminum salts such as aluminum hydroxide (alum) and aluminum phosphate. Alum is a component of numerous deactivated or subunit vaccines for tetanus, diphtheria, pertussis and hepatitis B virus vaccines, among others. In animal experiments it has been demonstrated that for influenzavirus vaccines the adjuvated antigens in split or subunit vaccines are superior to the corresponding fluid vaccines. Consequently, a human split vaccine adjuvated with alum was also developed. However, clinical studies showed no statistically significant difference in the seroconversion rate vis-a-vis the adjuvant-free influenza fluid vaccine (Lehmann, Die gelben Hefte [Yellow series], 21, 76-80 (1981)). Moreover, the adjuvated influenza vaccine involved an increased local vaccine reaction, so the adjuvation of influenza vaccines is generally not recommended; as a matter of fact, no human influenza vaccine adjuvated with alum has ever been on the market.

In clinical trials the immunogenicity and tolerance of an influenza subunit vaccine (Agrippal″) and Agrippal adjuvated with MF59 (Fluad®) were tested comparatively. It was shown that the adjuvated vaccine is safe and well-tolerated and the addition of MF59 to the vaccine increased the immunogenicity of the influenza vaccine, especially in the elderly, with low prevaccinal titers (De Donata et al. Vaccine 17, 3094-3101 (1999)).

The superiority of Fluad® was also shown compared to a nonadjuvated split vaccine (Menegon et al. Eur. J. Epidemiol. 15, 573-576 (1999)).

Fluad® was licensed in Italy in 1997 and has been commercially available in Italy since the flu season of 1997/1998. Because of its profile of action, Fluad® is especially advantageous for the following persons:

immunosuppressed patients (e.g. immunosuppression caused by high-dose steroid treatment, condition after transplantation, dialysis patients)

special risk groups such as diabetics

nursing home residents

As already mentioned above, this patient group for the most part overlaps the group that is at an increased risk of pneumococcus infection. The currently available pneumococcus vaccines consist mainly of purified capsule polysaccharides of the 23 most important serotypes of S. pneumoniae; e.g., the pneumococcus vaccines Pneumopur(® and Pneumovax 23® available in Germany did not show any protective effectiveness against pneumococcal pneumonia in most randomized, controlled clinical trials. Nonetheless, the pneumococcus vaccine is recommended in industrialized countries by the respective national ministries, medical associations and advising committees for the elderly, immunosuppressed adults and also children with chronic illnesses, among others (STIKO vaccine recommendations). Attempts to optimize the currently available pneumococcus vaccines have for the most part failed, since the vaccine's amount of antigen from polysaccharide and protein carrier would increase tremendously and the tolerance would be unsatisfactory. Moreover, the vaccine would be considerably more expensive than a pure polysaccharide vaccine due to the protein conjugate technology used in its manufacture. The direct addition of the adjuvant MF59 to the vaccine preparation could have unforeseen negative consequences and entail expensive research on the physicochemistry of the antigens, the immunity and the tolerance.

As the specialist knows, such drawbacks are of course also applicable to the addition of MF59 to all other vaccine preparations. A surprising alternative for optimizing the effectiveness of pre-existing vaccines, in particular pneumococcal polysaccharide vaccines or pneumococcal polysaccharide conjugate vaccines, now consists of the contralateral and simultaneous application of such vaccines with an influenza vaccine adjuvated with MF59. The adjuvant contained in the vaccine adjuvated with MF59 surprisingly increases not only the immunogenicity of the influenzavirus-specific antigen, but also the immunogenicity of the pneumococcal polysaccharide antigens and the polysaccharide conjugate, respectively. Moreover, the protective titer induced by the vaccine remains at a higher level for a longer time, so the interval between subsequent pneumococcus inoculations can be extended.

Additional preferred embodiments of the invention in question are the use of MF59-adjuvated protein subunit influenza vaccines in combination with a rabies vaccine for the post-exposure prophylaxis of rabies, the simultaneous contralateral application with tetanus or diphtheria vaccines, e.g. in patients weakened by hemodialysis, the simultaneous contralateral application with the HBV surface antigen or HIV antigens such as gp 120, the simultaneous contralateral application with a vaccine against early summer meningoencephalitis virus (ESME) and the simultaneous contralateral application with additional polysaccharide vaccines such as e.g. against typhus and meningococcus A and/or C, as well as other meningococcus serotypes.

1. Use of an oil-in-water emulsion as adjuvant for vaccines to be applied contralaterally, the emulsion comprising approx. 5% squalene, 0.5% polysorbate 80 and 0.5% sorbitan trioleate in aqueous citrate buffer pH 6.5.

2. Vaccine containing a first vaccine, adjuvated with an emulsion as defined in accordance with claim 1, and a nonadjuvated second vaccine as combination partners for the simultaneous, separate or phased application for immunization against viral, bacterial or parasitic infectious diseases.

3. Vaccine according to claim 2, the second vaccine being applied simultaneously and contralaterally.

4. Vaccine according to claim 2 or 3, the adjuvated vaccine being an influenza protein subunit vaccine.

5. Vaccine according to one of the claims 2, 3, or 4, the nonadjuvated vaccine being a pneumococcal capsule polysaccharide vaccine or a pneumococcal polysaccharide conjugate vaccine.

6. Vaccine according to one of the claims 2 to 5 for immunization against influenza and pneumococcus infections.

7. Vaccine according to one of the claims 2 to 6, the second vaccine being adjuvated with an aluminum compound.

8. Vaccine according to one of the claims 2, 3, 4, 6 or 7, the second vaccine being a nucleic acid vaccine.

9. Vaccine according to one of the claims 2, 3, 4, 6 or 7, the second vaccine being a whole-cell vaccine.

10. Vaccine according to one of the claims 2, 3, 4, 6 or 7, the second vaccine being a protein subunit vaccine.

11. Vaccine according to one of the claims 2 to 7, the second vaccine being a polysaccharide vaccine.

12. Vaccine according to one of the claims 2, 3, 4, 6, or 7, the second vaccine being a polysaccharide conjugate vaccine.

13. Vaccine according to one of the claims 2 to 12, the second vaccine being used therapeutically and/or after exposure.

14. Vaccine according to one of the claims 2 to 13, the second vaccine being a rabies vaccine.

15. Vaccine according to one of the claims 2, 3, 4, 7, 8, 9, 10, 11, 12, 13 or 14 for post-exposure rabies prophylaxis.

16. Vaccine according to one of the claims 2, 3, 4, 7, 8, 9, 10, 11, 12, or 13, the second vaccine being a diphtheria vaccine.

17. Vaccine according to one of the claims 2, 3, 4, 7, 8, 9, 10, 11, 12, or 13, the second vaccine being a tetanus vaccine.

18. Vaccine according to one of the claims 2, 3, 4, 7, 8, 9, 10, 11, 12, or 13, the second vaccine being a meningococcus vaccine against serotype A, C or other serotypes.

19. Vaccine according to one of the claims 2, 3, 4, 7, 8, 9, 10, 11, 12 or 13, the second vaccine being an HIV vaccine.

20. Vaccine according to one of the claims 2, 3, 4, 7, 8, 9, 10, 11, 12 or 13, the second vaccine being an HBV vaccine.

21. Vaccine according to one of the claims 2, 3, 4, 7, 8, 9, 10, 11, 12 or 13, the second vaccine being a Helicobacter pylori vaccine.

22. Vaccine according to one of the claims 2, 3, 4, 7, 8, 9, 10, 11, 12 or 13, the second vaccine being an early summer meningoencephalitis vaccine (ESME).

23. Vaccine according to one of the claims 2, 3, 4, 7, 8, 9, 10, 11, 12 or 13, the second vaccine being a typhus vaccine.
